(19) United States
(12) Patent Application Publication
(10) Pub. No.: US 2024/0350321 A1
(43) Pub. Date: Oct. 24, 2024

(54) BANDAGE WITH ADHESIVE LAYER HAVING VARYING PERFORATION SHAPES AND SIZES

(71) Applicant: Biodaptive Advanced Materials, LLC, Ronkonkoma, NY (US)

(72) Inventors: Daniel Rivest, Miami, FL (US); Michael R. Bielski, Manorville, NY (US)

(21) Appl. No.: 18/684,394

(22) PCT Filed: Aug. 18, 2022
(86) PCT No.: PCT/US2022/040754

§ 371 Date: Feb. 16, 2024

Related U.S. Application Data
(60) Provisional application No. 63/325,370, filed on Mar. 30, 2022.
(60) Provisional application No. 63/282,740, filed on Nov. 24, 2021.
(60) Provisional application No. 63/234,504, filed on Aug. 18, 2021.

(51) Int. Cl. A61F 13/0246; A61F 13/0203

(52) U.S. Cl. CPC A61F 13/025; A61F 13/0203

(57) ABSTRACT

A bandage with an adhesive layer including perforations of different sizes, shapes and spacings between them.

### CROSS-REFERENCE TO RELATED APPLICATIONS

The present application claims benefit of and priority to U.S. Provisional Patent Application No. 63/325,370 filed Mar. 30, 2022 entitled BANDAGE WITH ADHESIVE LAYER HAVING VARYING PERFORATION SHAPES AND SIZES, U.S. Provisional Patent Application Ser. No. 63/282,740 filed Nov. 24, 2021 entitled BANDAGE WITH ADHESIVE LAYER HAVING VARYING PERFORATION SHAPES AND SIZES and U.S. Provisional Patent Application Ser. No. 63/234,504 filed Aug. 18, 2021 entitled BANDAGE WITH ADHESIVE LAYER HAVING VARYING PERFORATION SIZES, the entire content of each of which is incorporated by reference herein.

### BACKGROUND

### Field of the Disclosure

The present invention relates to a bandage including a perforated adhesive layer including variably sized, shaped and spaced perforations.

### Related Art

Bandages including absorbent material are typically provided to cover wounds while they heal. Bandages may also be used as a preventative measure to prevent formation of sores or wounds. Conventional bandages typically include openings in an adhesive layer that contacts the user's skin to allow exudate from the wound to be drawn away from the wound site and absorbed in an absorbent layer. The inclusion of these perforations allows fluid to be absorbed into the bandage, however, may negatively affect adhesion of the bandage to the user's skin.

Accordingly, it would be beneficial to provide a bandage including an adhesive layer that optimizes absorption of fluid from the wound while providing improved adhesion to the user's skin.

### SUMMARY

It is an object of the present disclosure to provide a bandage that optimizes absorption of fluid from a wound covered by the bandage while improving adhesion to the user's skin.

A bandage in accordance with an embodiment of the present disclosure includes: a top film; an absorbent layer positioned below the top film; an adhesive layer provided below the absorbent layer; wherein the adhesive layer includes: a layer of adhesive provided on a bottom surface of the adhesive layer; at least one central opening provided in a central portion of the adhesive layer, the at least one central opening having a first size and a first shape configured to maximize fluid passage into the absorbent layer; and at least one peripheral opening provided in a peripheral portion of the adhesive layer, the at least one peripheral opening having a second size and a second shape configured to maximize adhesion of the adhesive layer to a user's skin.

In embodiments, the absorbent layer includes an upper layer adhered to the top film and a lower layer adhered to the adhesive layer.

In embodiments, the absorbent layer includes a superabsorbent layer.

In embodiments, the first size of the at least one central opening is larger than the second size of the at least one peripheral opening.

In embodiments, the at least one central opening includes a first plurality of central openings.

In embodiments, each central opening of the first plurality of central openings has the first shape.

In embodiments, at least a first group of central openings of the first plurality of central openings have the first shape and a second group of central openings of the first plurality of central openings have a third shape different from the first shape.

In embodiments, at least a first group of central openings of the first plurality of central openings are arranged in a first pattern.

In embodiments, at least a second group of central openings of the first plurality of central openings are arranged in a second pattern.

In embodiments, the second pattern is the same as the first pattern.

In embodiments, the second pattern is different from the first pattern.

In embodiments, at least one central opening of the first plurality of central openings is larger than at least one other central opening of the first plurality of central openings.

In embodiments, a space between the first plurality of central openings varies to maximize fluid passage into the absorbent layer.

In embodiments, the at least one central opening comprises a curved slot.

In embodiments, the at least one peripheral opening comprises a second plurality of central openings.

In embodiments, each peripheral opening of the second plurality of central openings has the second shape.

In embodiments, at least a third group of peripheral openings of the second plurality of peripheral openings have the second shape and a fourth group of peripheral openings of the second plurality of peripheral openings have a fourth shape different from the second shape.

In embodiments, at least one peripheral opening of the second plurality of peripheral openings is larger than at least one other peripheral opening of the second plurality of peripheral openings.

In embodiments, at least a third group of central openings of the second plurality of peripheral openings are arranged in a third pattern.

In embodiments, at least a fourth group of central openings of the second plurality of peripheral openings are arranged in a fourth pattern.

In embodiments, the fourth pattern is the same as the third pattern.

In embodiments, the fourth pattern is different from the third pattern.

In embodiments, a space between the second plurality of peripheral openings varies to minimize adhesion.

In embodiments, the at least one peripheral opening comprises a curved slot.

In embodiments, the adhesive layer includes a perforation free region positioned between the at least one central opening and the at least one peripheral opening that does not include any openings.

### BRIEF DESCRIPTION OF THE DRAWINGS

The above and related objects, features and advantages of the present disclosure will be more fully understood by reference to the following, detailed description of the preferred, albeit illustrative, embodiments of the present invention when taken in conjunction with the accompanying figures, wherein:

FIG. 1 illustrates an exploded view of a bandage in accordance with an exemplary embodiment of the present disclosure;

FIG. 2 illustrates a more detailed view of a bottom, skin facing surface of the bandage of FIG. 1;

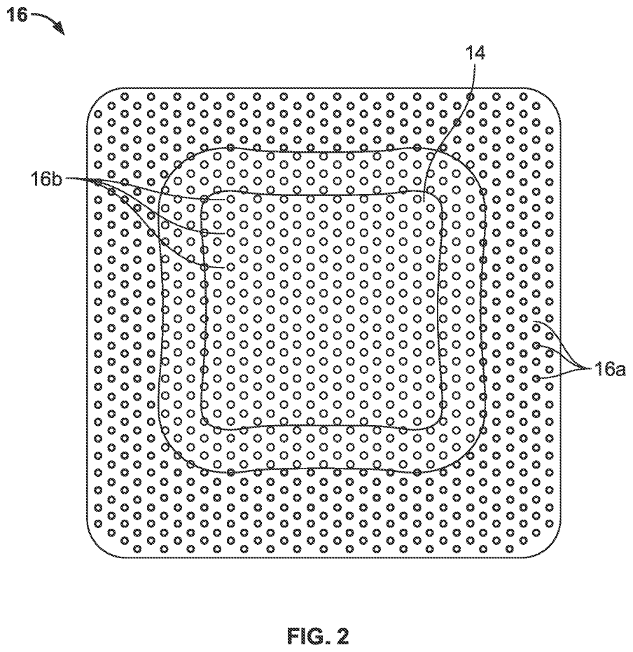

FIGS. 3A-3D illustrate different perforation shapes that may be used in the bandage of FIG. 1;

FIGS. 4A-4E illustrate exemplary groupings of perforations that may be used in the bandage of FIG. 1;

FIGS. 5A-5F illustrate exemplary perforation patterns that may be used in the bandage of FIG. 1; and

FIG. 6 illustrates an exemplary graph showing the relationship between surface area in contact with the user's skin, adhesion and permeability; and

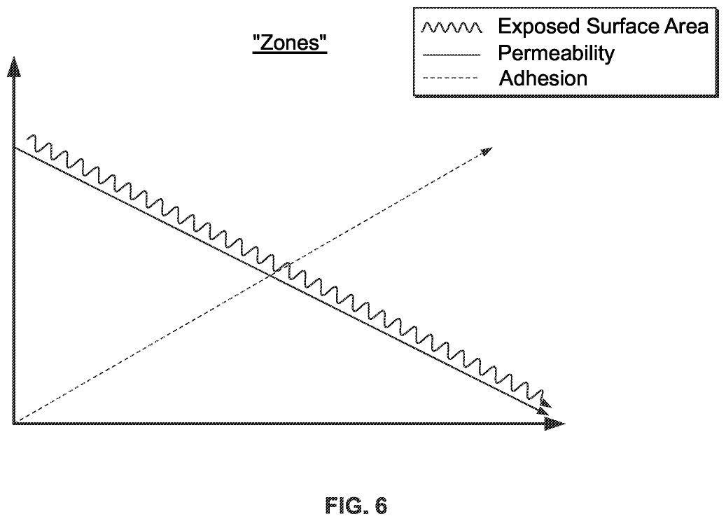

FIG. 7 an exploded view of a bandage in accordance with another exemplary embodiment of the present disclosure.

### DETAILED DESCRIPTION OF THE EXEMPLARY EMBODIMENTS

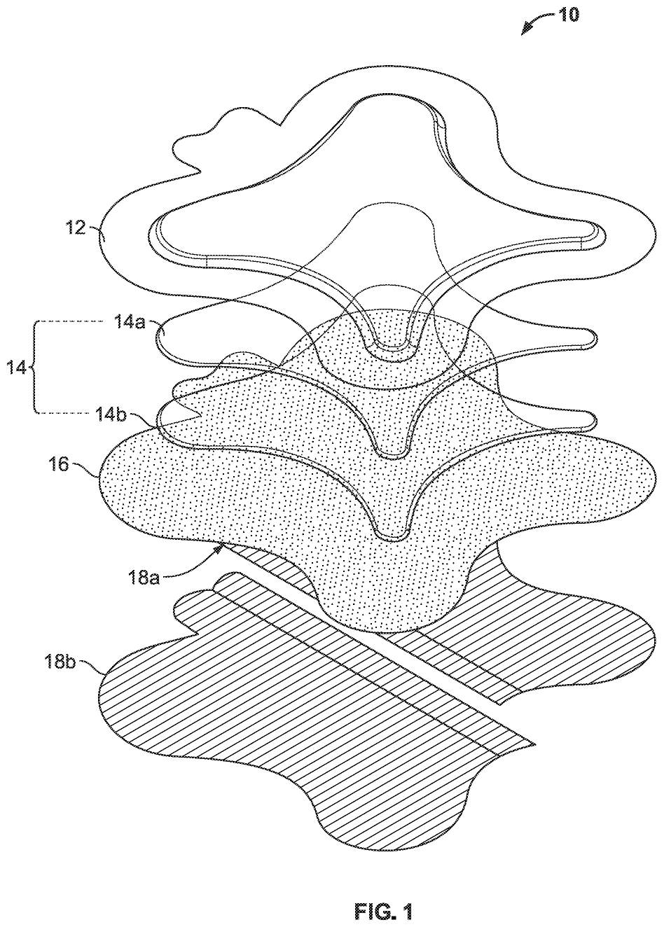

A bandage 10 in accordance with an embodiment of the present disclosure is illustrated in FIG. 1. In embodiments, the bandage 10 includes a top film 12. In embodiments, this top film 12 may be made of a material that is impervious to fluid and air to protect a wound covered by the bandage 10. In embodiments, the top film 12 may be opaque or may be partially or fully transparent or translucent. In embodiments, the top layer or film 12 may be made of polyurethane. In embodiments, the top film 12 may allow for evaporation of fluid from the bandage 10. In embodiments, an absorbent layer 14 may be provided under the top film 12 on a side closer to the user's skin when the bandage 10 is in place. In embodiments, the absorbent layer 14 may be adhered to the top film 12. In embodiments, the absorbent layer may be movable relative to the film 12. In embodiments, the absorbent layer 14 may be made of one or more of foam, such as medical grade polyurethane foams, superabsorbent polymers, superabsorbent fibers such as or alginate gelling fiber, to name a few. In embodiments, the absorbent layer 14 may be made of, or include, a hydrogel or a hydrocolloid. In embodiments, the absorbent layer may be made of a fiber material. In embodiments, the absorbent layer 14 may be made of any material that absorbs and transports exudate from the wound and/or moisture from the skin. In embodiments, the absorbent layer 14 may include a first, or top layer 14a connected to or otherwise positioned adjacent to the top film 12, and a second, or lower layer 14b positioned below the top layer 14a, closer to the user's skin when the bandage 10 is in place on the user's body. In embodiments the first layer 14a and second layer 14b may be made of the same material. In embodiments, the first layer 14a and the second layer 14b may be made of different materials. In embodiments, the first layer 14a and second layer 14b may be made of a combination of foam and a superabsorbent material. In embodiments, additional or fewer layers of absorbent material may be used in the absorbent layer 14. In embodiments, absorbency may be measured following the BS EN 13726-1 method and absorbency may be in the range of 0.05 g/cm² to 3 g/cm².

In embodiments, a silicone adhesive layer 16 may be provided under the absorbent layer 14 and may be positioned to be in contact with the user's skin when the bandage is in place on the user's body. In an embodiment, the adhesive layer 16 may be implemented using a clear polyurethane film with a silicone based adhesive provided at least on a bottom surface thereof to secure the bandage 10 to the user's skin. In embodiments, the adhesive layer 16 may be adhered to the absorbent layer 14. In embodiments, where the absorbent layer 14 includes the first and second absorbent layer 14a, 14b, the adhesive layer 16 may be adhered to the second, lower absorbent layer 14b.

In embodiments, the silicone adhesive of the adhesive layer 16 adheres to the user's skin around the wound while avoiding a strong bond with the wound. In embodiments, the silicone adhesive layer 16 may include a plurality of perforations 16a, 16b formed therethrough. In embodiments, the perforations 16a, 16b may be provided to allow exudate from the wound to pass through the adhesive layer 16 into the absorbent layer 14. In embodiments, a first set of perforations 16a may be positioned around the periphery of the bandage 10 and may have a first size which is smaller than a second size of the second set of perforations 16b positioned in an inner area of the bandage 10 which may be positioned over the wound. In embodiments, the first set of perforations 16a are between 0.3-0.7 mm, while the second set of perforations 16b are between 1.1-1.5 mm. In embodiments, the first set of perforations 14a are provided around a periphery of the bandage 10 and typically outside the borders of the wound. The smaller size of these perforations 14a maximizes the surface area in contact with the skin and allows for a strong bond between the bandage 10 and the user's skin surrounding the wound while still allowing any fluid around the periphery of the wound to pass out through the openings 14a. In embodiments, the second set of perforations 16b are formed in a central portion of the bandage adjacent to the absorbent layer 14 and over the wound such that exudate from the wound passes through the perforations 16b into the absorption layer 14. In embodiments, the larger size of the perforations 16b allows for more exudate to pass through the perforations 16b and into the absorbent layer 14 from the wound. In embodiments, the larger size of the perforations 16b reduces contact between the adhesive and the wound such that the bandage 10 will release from the wound when necessary. In embodiments, the adhesive layer 16 and the film 12 extend outward beyond the periphery of the absorbent layer 14. In embodiments, other materials may be used to make the film in the adhesive layer 16, provided that the adhesive that faces the user's skin is silicone based. In embodiments, any adhesive that is suitable for contact with a user's skin may be used. In embodiments, the perforations 16a, 16b in the adhesive layer 16 allow exudate to pass therethrough and into the absorbent layer 14. The silicone adhesive layer 16 provides a stable and firm connection to the user's skin to keep the bandage 10 in place while being relatively comfortable. In addition, the silicone adhesive allows for relatively easy removal, without damaging the wound.

In embodiments, one or more protective liners 18a, 18b may be provided to cover the perforated silicone adhesive lower layer 16 prior to use. In embodiments, the protective liners 18a, 18b are preferably removable from the adhesive layer 16 and are preferably made of polyethylene. In embodiments, the liners 18a, 18b may be made of a coated paper material. In embodiments, the liners 18a, 18b may be made of other materials, provided that they allow them to be removable from the adhesive layer 16 without damaging the adhesive.

In embodiments, a packaging element may be provided to store the bandage 10 before use. In embodiments, the packaging element may be made of a combination of paper and polyethylene, or paper and forms a pouch or pocket in which the bandage 10 may be stored. In embodiments, the packaging element may be made of any other suitably durable material. In embodiments, the packaging element may be made of a material that is suitable to allow for sterilization of the bandage 10 while in place in the packaging element. In embodiment the packaging element may be made of a material that is impervious to liquids. In embodiments, the packaging element may be made of a material that allows liquid to exit, but not enter, or that allows liquid to enter but not exit. In embodiments, the packaging element may be transparent or include transparent portions. In embodiments, the packaging element may include an upper part and a lower part, with the bandage 10, including the layers discussed above, sandwiched between the upper and lower parts.

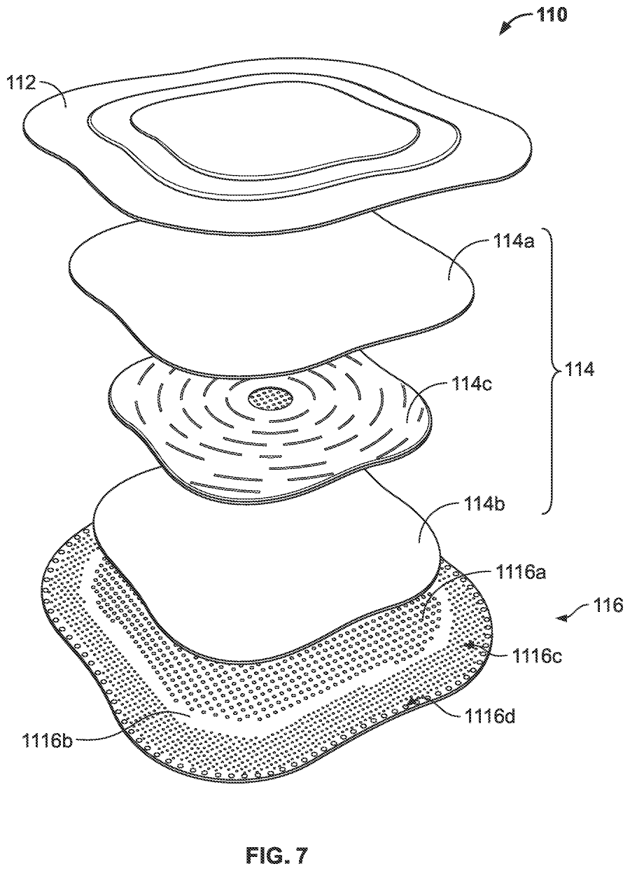

In embodiments, the shape, size, spacing and/or orientation of the perforations 16a, 16b may be varied in order to maximize adhesion between the bandage 10 and the user's skin around the wound while allowing for optimal permeability to draw fluid away from the wound covered by the bandage. In general, optimal permeability is desired in the center portion of the bandage 10 that covers and contacts the wound to allow fluid to be drawn away and promote healing, while adhesion is preferably optimized around the edges of the bandage, which extend beyond the border of the wound to promote a strong connection between the bandage 10 and the user's skin outside of the wound area so that the bandage is securely held in place. In general, the more surface area of the adhesive layer 16 that contacts the user's skin, the stronger the adhesion. As a result, small perforations maximize surface area in contact with the user's skin and generally provide for better adhesion. Small perforations, however, may limit the amount of exudate that may pass through the adhesive layer 16, and thus, tend to result in lower permeability of the adhesive layer 16. Larger perforations, which result in less surface area in contact with the user's skin, tend to result in increased permeability, but reduced adhesion. FIG. 7 illustrates an exemplary graph illustrating the general relationship of surface area, permeability and adhesion. In general, as can be seen with reference to FIG. 7, as surface area decreases, so does permeability, however, adhesion increases. Varying the size, shape and positioning of the perforations may be used to optimize permeability and adhesion for different portions of the bandage 10.

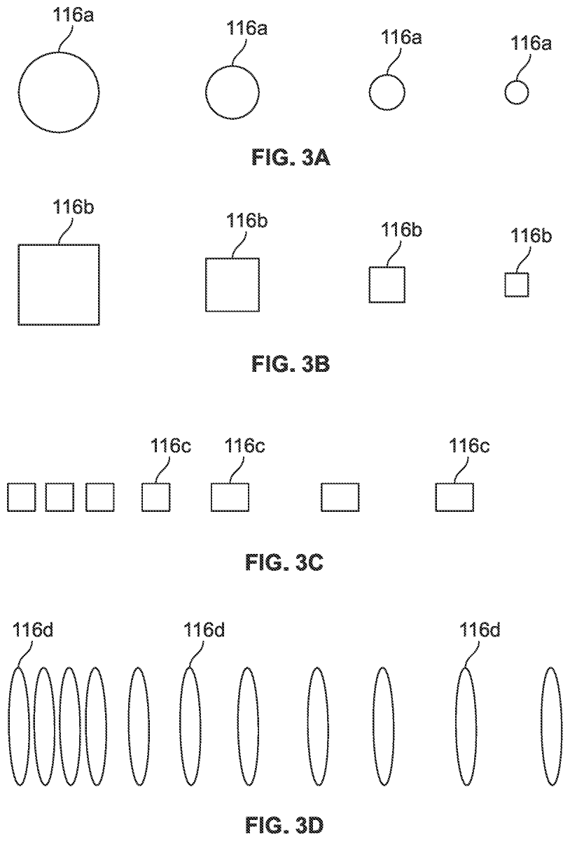

In embodiments, the shape of the perforations 16a, 16b provided in the adhesive layer 16 may be varied, which may vary the effective surface area of the adhesive layer 16 in contact with the user. FIG. 3A illustrates an exemplary embodiment in which perforations 116a are circular. The perforations 116a may be used in place of or in addition to the perforations 16a. As illustrated in FIG. 3A, the size of the circular perforations 116a may be varied and larger perforations may be used in place of or in addition to the perforations 16b. In embodiments, a variety of different sized circular perforations may be used in the adhesive layer 16 to optimize permeability and adhesions in different portions. In embodiments, as shown in FIG. 3B, perforations 116b may be square shaped and may be used in place of or in addition to the perforations 16a. In embodiments, the size of the square shaped perforations 116b may be varied as can be seen in FIG. 3B and larger sized perforations may be used in place of or in addition to the perforations 16b. In embodiments, a variety of sizes of square shaped perforations may be provided in the adhesive layer 16 to optimize permeability and adhesion. In embodiments, rectangular shaped perforations 116c may be provided in the adhesive layer 16, such as those illustrated in FIG. 3C, for example, in place of or in addition to the perforation 16a. As can be seen in FIG. 3C, the rectangular shaped perforations 116c may be provided in larger sizes which may be used in place of or in addition to the perforations 16b. In embodiments, the adhesive layer 16 may include a variety of sizes of rectangular perforations 116c at different positions to optimize permeability and adhesion. In embodiments, oval shaped perforations 116d may be provided in the adhesive layer 16 in place of or in addition to the perforations 16a. In embodiments, the oval shaped perforations 116d may have larger sizes which may be used in place of or in addition to the perforations 16b. In embodiments, a variety of sizes of oval shaped perforations 116d may be provided in the adhesive layer.

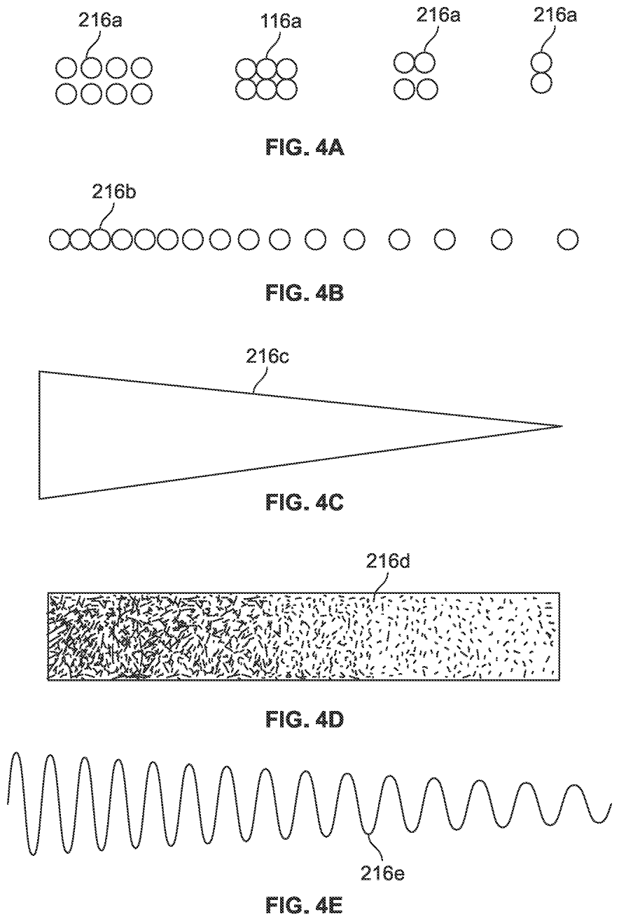

In embodiments, circular shaped perforations 216a may be provided in the adhesive layer 16 and clustered in groups as can be seen in FIG. 4A, for example. In embodiments, each group may include the same number of perforations. In embodiments, different numbers of perforations may be included in one or more groups. In embodiments, circular shaped perforations 216b may be provided in the adhesive layer 16 with varying spacing provided between the perforations, as shown in FIG. 4B, for example. Varying the spacing between the perforations 216b may be used to change the effective surface area in contact with user's skin at different positions. In embodiments, perforations of other shapes may be clustered.

In embodiments, one or more perforations 216c may have a wedge shape and may be provided in the adhesive layer 16 as can be seen in FIG. 4C, for example. In embodiments, spacing between perforations 216d of similar size may be varied in order to adjust the amount of surface area in contact with the user's skin as can be seen in FIG. 4D, for example. In embodiments, a slot or channel 216e may be formed in the adhesive layer 16 as can be seen on FIG. 4E, for example. In embodiments, the slot or channel 216e may have a curved shape which may vary in order to provide different effective surface areas in contact with the user's skin at different points in the bandage to vary permeability and adhesion as desired.

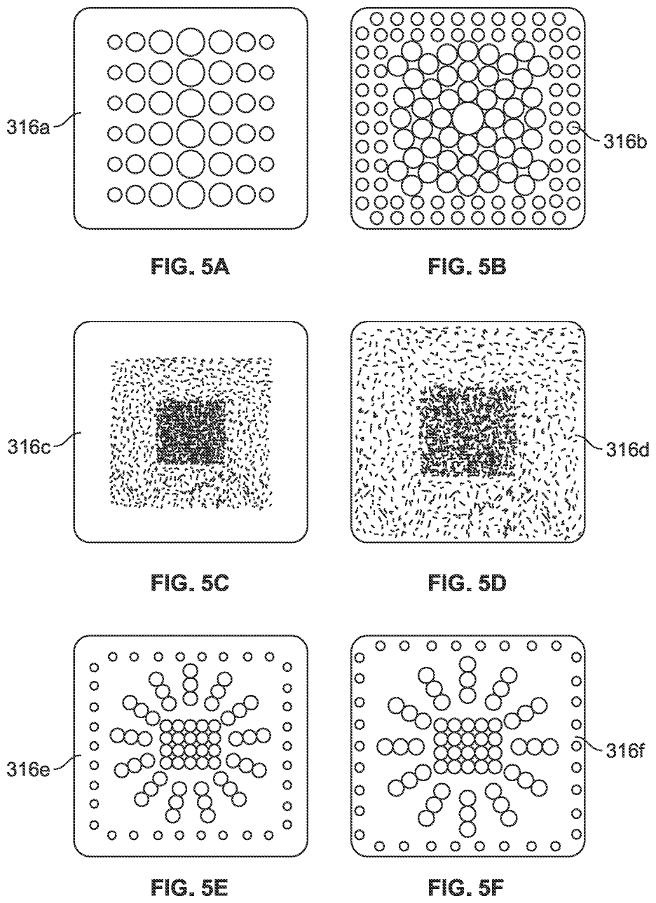

In embodiments, since the periphery of the bandage 10, and the adhesive layer 16 is generally positioned outside of the wound boundary, there may be little to no need to provide perforations in this area since there is no or minimal exudate being expressed in the area. FIGS. 5A, 5C and 5E illustrate patterns of perforations, 316a, 316c, 316e in which there are no perforations provided around a periphery of the adhesive layer 16. The interior area of the embodiment in FIG. 5A includes circular perforations 316a of different sizes, with larger sizes provided in the middle, to maximize permeability and minimize adhesion directly over the wound. In FIG. 5C, the perforations 316c in the interior have substantially the same size, however, the spacing between them is varied to change the effective surface area in contact with the user with minimal spacing in the center over the wound to allow increased permeability and decreased adhesion and larger spacing extending outward from the middle, resulting in increased surface area in contact with the user. FIG. 5E illustrates perforations 316e in the interior that are grouped in order to optimize permeability in a center or middle and increase adhesion extending away from the center.

FIGS. 5B, 5D and 5F illustrate perforations 316b, 316d and 316f that are provided around the periphery of the adhesive layer as well as in the interior. In FIG. 5B, the perforations 316b around the periphery have a relatively small size and the perforations in the interior have varying, but generally larger sizes to optimize permeability in the middle and adhesion around the periphery. In FIG. 5D, the perforations 316d are more or less evenly spaced around the periphery while the spacing between perforations 316d is varied in the interior such that spacing in the middle is minimized to reduce effective surface area and increase permeability and spacing increases away from the middle to increase adhesion. The perforations 316f in FIG. 5F include a regular distribution of circular perforations around the periphery to provide consistent adhesion and varying groupings of perforations provided in the interior to optimize permeability in the middle and increase adhesion extending away from the center.

FIG. 7 illustrates a bandage 110 in accordance with an embodiment of the present disclosure. In embodiments, the bandage 110 may include a top film 112 which may be similar to the top film 12 discussed above. In embodiments, an absorbent layer 114 may be provided under the top film 112 on a side closer to the user's skin when the bandage 110 is in place. In embodiments, the absorbent layer 114 may be adhered to the top film 112. In embodiments, the absorbent layer 114 may be movable relative to the film 112. In embodiments, the absorbent layer 114 may be made of one or more of foam, such as medical grade polyurethane foams, superabsorbent polymers, superabsorbent fibers such as or alginate gelling fiber, to name a few. In embodiments, the absorbent layer 114 may be made of, or include, a hydrogel or a hydrocolloid. In embodiments, the absorbent layer 114 may be made of a fiber material. In embodiments, the absorbent layer 114 may be made of any material that absorbs and transports exudate from the wound and/or moisture from the skin. In embodiments, the absorbent layer 114 may include a first, or top layer 114a connected to or otherwise positioned adjacent to the top film 112, and a second, or lower layer 114b positioned below the top layer 114a, closer to the user's skin when the bandage 110 is in place on the user's body. In embodiments, the first layer 114a and second layer 114b may be made of the same material. In embodiments, the first layer 114a and the second layer 114b may be made of different materials. In embodiments, an intermediate layer 114c may be provided between the first layer 114a and the second layer 114b may be made of a superabsorbent material. In embodiments, absorbency may be measured following the BS EN 13726-1 method and absorbency may be in the range of 0.05 g/cm² to 3 g/cm².

In embodiments, the first layer 114a or the second layer 114b may be made of superabsorbent material. In embodiments, the intermediate layer 114c may be made of any other absorbent material. In embodiments, as illustrated in FIG. 7, the absorbent layer 114 may be slightly smaller than the top film 112 such that the top film extends beyond the edges of the absorbent layer 114 and the adhesive layer 116 extends beyond the periphery thereof as well. In embodiments, the intermediate layer 114c may be made of a superabsorbent gel. In embodiments, the intermediate layer 114c may include one or more perforations 114d to add flexibility. In embodiments, additional or fewer layers of absorbent material may be used in the absorbent layer 114.

In embodiments, a silicone adhesive layer 116 may be provided under the absorbent layer 114 and may be positioned to be in contact with the user's skin like the adhesive layer 16. In embodiments, the adhesive layer 116 may be implemented using a clear polyurethane film with a silicone based adhesive provided at least on a bottom surface thereof to secure the bandage 110 to the user's skin like the adhesive layer 16. In embodiments, the adhesive layer 116 may be adhered to the absorbent layer 114, for example, the second, lower absorbent layer 114b. In embodiments, the adhesive layer 1116 may include a variety of perforations. In embodiments, a central portion of the absorbent layer 116 may include large openings 1116a to allow exudate from the wound to pass through and into the adhesive layer 114. In embodiments, a perforation free zone 1116b surrounds the large openings 1116a to provide for improved adhesion to the user's skin. In embodiments, this perforation free zone 1116b is preferably provided such that it is outside the boundary of the wound when the bandage 110 is in place and provides a seal around the periphery of the wound. In embodiments, a small perforation zone 1116c may be provided around the perforation free zone 1116b. In embodiments, the small perforation zone 1116c may include small perforations, smaller than the large opening 1116a such that the increased surface area allows for better adhesion to the user's skin. In embodiments, a larger perforation zone 1116d may be provided around the small perforation zone 1116d on the periphery of the bandage 110. In embodiments, the large openings of this larger perforation zone 1116d may be aligned with the edges of the top film 112 such that adhesive on a bottom of the top film 112 may adhere to the user's skin and provide an additional seal around the bandage 110.

Although the present invention has been described in relation to particular embodiments thereof, many other variations and modifications and other uses will become apparent to those skilled in the art. It is preferred, therefore, that the present invention be limited not by the specific disclosure herein.

What is claimed is:

1. A bandage comprises:
a top film;
an absorbent layer positioned below the top film;
an adhesive layer provided below the absorbent layer;
wherein the adhesive layer includes:
a layer of adhesive provided on a bottom surface of the adhesive layer;
at least one central opening provided in a central portion of the adhesive layer, the at least one central opening having a first size and a first shape configured to maximize fluid passage into the absorbent layer; and
at least one peripheral opening provided in a peripheral portion of the adhesive layer, the at least one peripheral opening having a second size and a second shape configured to maximize adhesion of the adhesive layer to a user's skin.

2. The bandage of claim 1, wherein the absorbent layer comprises an upper layer adhered to the top film and a lower layer adhered to the adhesive layer.

3. The bandage of claim 1, wherein the absorbent layer includes a superabsorbent layer.

4. The bandage of claim 1, wherein the first size of the at least one central opening is larger than the second size of the at least one peripheral opening.

5. The bandage of claim 1, wherein the at least one central opening comprises a first plurality of central openings.

6. The bandage of claim 5, wherein each central opening of the first plurality of central openings has the first shape.

7. The bandage of claim 5, wherein at least a first group of central openings of the first plurality of central openings have the first shape and a second group of central openings of the first plurality of central openings have a third shape different from the first shape.

8. The bandage of claim 5, wherein at least a first group of central openings of the first plurality of central openings are arranged in a first pattern.

9. The bandage of claim 8, wherein at least a second group of central openings of the first plurality of central openings are arranged in a second pattern.

10. The bandage of claim 9, wherein the second pattern is the same as the first pattern.

11. The bandage of claim 9, wherein the second pattern is different from the first pattern.

12. The bandage of claim 5, wherein at least one central opening of the first plurality of central openings is larger than at least one other central opening of the first plurality of central openings.

13. The bandage of claim 5, wherein a space between the first plurality of central openings varies to maximize fluid passage into the absorbent layer.

14. The bandage of claim 5, wherein the at least one central opening comprises a curved slot.

15. The bandage of claim 1, wherein the at least one peripheral opening comprises a second plurality of central openings.

16. The bandage of claim 15, wherein each peripheral opening of the second plurality of central openings has the second shape.

17. The bandage of claim 15, wherein at least a third group of peripheral openings of the second plurality of peripheral openings have the second shape and a fourth group of peripheral openings of the second plurality of peripheral openings have a fourth shape different from the second shape.

18. The bandage of claim 15, wherein at least one peripheral opening of the second plurality of peripheral openings is larger than at least one other peripheral opening of the second plurality of peripheral openings.

19. The bandage of claim 15, wherein at least a third group of central openings of the second plurality of peripheral openings are arranged in a third pattern.

20. The bandage of claim 19, wherein at least a fourth group of central openings of the second plurality of peripheral openings are arranged in a fourth pattern.

21. The bandage of claim 20, wherein the fourth pattern is the same as the third pattern.

22. The bandage of claim 20, wherein the fourth pattern is different from the third pattern.

23. The bandage of claim 15, wherein a space between the second plurality of peripheral openings varies to minimize adhesion.

24. The bandage of claim 1, wherein the at least one peripheral opening comprises a curved slot.

25. The bandage of claim 1, wherein the adhesive layer includes a perforation free region positioned between the at least one central opening and the at least one peripheral opening that does not include any openings.
